(19) United States
(12) Patent Application Publication
(10) Pub. No.: US 2025/0271419 A1
(43) Pub. Date: Aug. 28, 2025

(54) PLACENTA ORGANOID ON-A-CHIP AND USE THEREOF

(71) Applicant: THE REGENTS OF THE UNIVERSITY OF CALIFORNIA, Oakland, CA (US)

(72) Inventors: Quinton Smith, Irvine, CA (US); Melisa J. Andrade, Irvine, CA (US); Eric Wang, Irvine, CA (US)

(21) Appl. No.: 19/067,258

(22) Filed: Feb. 28, 2025

Related U.S. Application Data
(60) Provisional application No. 63/558,892, filed on Feb. 28, 2024.

(51) Int. Cl. G01N 33/50; C12N 5/073

(52) U.S. Cl. CPC G01N 33/5014; C12N 5/0605; G01N 33/5082; C12N 2501/11; C12N 2501/115; C12N 2506/45

(57) ABSTRACT

Methods, systems, and kits for generating placenta-like organoids in suspension or placenta-on-a-chip systems are described herein, as well as methods of use thereof. A placental organoid composition has a plurality of trophoblast cells derived from naïve-like stem cells in suspension cultures. The placental organoid composition can be prepared by seeding a single-cell suspension onto a culture surface in a first medium, centrifuging and incubating the single-cell suspension to form aggregates. A portion of the first media is replaced with a differentiation media and the aggregates are incubated for a period of time during which the aggregates differentiate into placental organoids. The composition can be used to assess drug toxicity and drug screening.

### CROSS-REFERENCE TO RELATED APPLICATIONS

This application is a non-provisional and claims benefit of U.S. Provisional Application No. 63/558,892 filed Feb. 28, 2024, the specification of which is incorporated herein in their entirety by reference.

### FIELD OF THE INVENTION

The present invention features placenta-like organoids (e.g., organoids on a chip) and uses thereof.

### BACKGROUND OF THE INVENTION

The human placenta is a multifunctional extraembryonic organ that forms an essential component of the maternal-fetal interface, facilitating the nourishment, protection, and health of the developing fetus. Structurally, the placenta is heterogeneous and derived from the trophoectoderm and the extraembryonic mesoderm. Originating from the trophoectoderm layer of the preimplantation embryo, the trophoblast lineages form the major cell types in the placenta. Among the trophoblasts, cytotrophoblasts (CTBs) are self-renewing multipotent progenitor cells that give rise to either syncytiotrophoblasts (STBs) by cell-cell fusion in chorionic villi or differentiate into extravillous trophoblasts (EVTs). While the STBs secrete various growth factors and hormones such as chorionic gonadotropin (hCG), placental growth hormone, estrogen, and progesterone, the EVTs migrate and invade the uterine wall, thereby anchoring the placenta onto the uterus and remodeling the maternal spiral arteries to establish blood supply to the fetus. Impairment in placental development leads to pregnancy complications such as fetal growth restriction, preeclampsia, pre-term birth, stillbirth, and miscarriage. However, the lack of accessibility to early-stage developing human placental tissues and physiologically relevant in vitro models has hampered the in-depth investigation.

Most of the current knowledge of placentation originates from animal models, immortalized trophoblasts, explanted term-human placental tissue or from unsuccessful pregnancies, tumor-derived or gestational choriocarcinoma. Although efforts have been made to develop animal models to mimic human placentation and implantation, there are considerable differences in developmental trajectories, degree of trophoblast invasion, and the number of cell layers at the maternal-fetal interface. The immortalized cell lines possess unnatural invasive behavior, harbor genetic abnormalities and are generated from a variety of sources with different phenotypes unable to represent all the trophoblast subtypes and features of in vivo placental tissue. Ethical constraints have limited access to explanted human placenta tissues from different gestational stages. Therefore, most of these model systems do not prove to be ideal for the study of human placentation. Furthermore, in comparison to conventional 2D systems, 3D organoids provide a more representative depiction of in vivo systems with regard to cell composition, structural organization, and biological functions. The recent development of induced pluripotent stem cell (iPSC)-derived placental organoids has led to the modeling of human development in vitro. Initial reports suggested the generation of placental organoids from only naïve stem cells, but reports also suggest direct differentiation from iPSCs. However, these methods often use hydrogel encapsulation, such as Matrigel, for the structural as well as functional development of the tissues, which often complicates their translational applications. Despite several advancements in the generation of model systems to study human placentation, disease states, or drug toxicity, currently, there are no physiologically relevant controllable in vitro models.

To address these limitations, the present invention features placenta-like organoids from naïve stem cells in suspension cultures without the utilization of hydrogels. A comparative analysis of the 2D, placenta-like organoids in suspension, and basement membrane extract (BME/Matrigel) encapsulation methods for the generation of placenta- like organoids has been performed to ascertain the resultant biological and functional relevance of organoids derived by each of these methods. These placenta-like organoids comprise heterogeneous cell types similar to the in vivo human placenta and expressed characteristic trophoblast markers representing the cytotrophoblast (CTB), syncytiotrophoblasts (STB), and extravillous trophoblasts (EVT) cell states. Furthermore, transcriptome analysis provided insights into the trophoblast development trajectory and the cell phenotypes in these stage-specific placental organoids. Secretome analysis of placenta-like organoids revealed the secretion of cytokines, hormones, and angiogenesis modulating factors as assessed in microfluidic devices, thereby providing a platform for investigating angiogenesis and vasculogenesis. Furthermore, given the lack of reliable platforms for the assessment of drug toxicity and implantation in pregnant women or early developing fetuses, the present invention provides proof-of-concept evidence that these systems can be used to evaluate drug toxicity and implantation disorders. Overall, the placenta-like organoids in suspension or placenta-on-a-chip systems will be a versatile tool to advance understanding of human placentation in vitro, model placental disorders, and test drug responses.

### BRIEF SUMMARY OF THE INVENTION

It is an objective of the present invention to provide compositions, methods, and kits that allow for development of placental organoids, enabling the in vitro recapitulation of placental tissue architecture and function, as specified in the independent claims. Embodiments of the invention are given in the dependent claims. Embodiments of the present invention can be freely combined with each other if they are not mutually exclusive.

In some embodiments, the present invention encompasses placental organoids for use in suspension culture or placenta-on-a-chip systems. The placental organoids described herein provide a versatile platform for modeling placental function, drug screening, and disease modeling in both static and dynamic environments.

In some embodiments, the present invention features a placental organoid composition comprising a plurality of trophoblast cells derived from naïve-like stem cells in a suspension culture. In some embodiments, the organoid exhibits a three-dimensional architecture resembling human placental tissue. Additionally, in some embodiments, the organoid demonstrates functional properties characteristic of human placental tissue, including expression of human leukocyte antigen-G (HLA-G), and the secretion of pregnancy related hormones (e.g., growth differentiation factor 15 (GDF15), Human Chorionic Gonadotropin (hCG), or a combination thereof).

In other embodiments, the present invention may also include methods for screening and assessing drug toxicity. In some embodiments, the method comprises administering a drug to the placental organoid composition as described herein for a period of time and measuring cell viability within the placental organoid composition using a viability assay. In some embodiments, the drug is toxic if the cell viability is reduced below a predetermined threshold. In certain embodiments, the drug is toxic if the cell viability is reduced to less than 50%. In other embodiments, the drug is toxic if the cell viability is reduced to less than 75%.

In further embodiments, the present invention may feature a method of generating a placental organoid composition. In some embodiments, the method comprises obtaining a single-cell suspension of naïve-like stem cells, seeding the single-cell suspension onto a culture surface in a first medium, centrifuging the single-cell suspension to facilitate aggregate formation, incubating the single cell suspension to form aggregates, and replacing at least a portion of the first media with a differentiation media and incubating the aggregates for a period of time. In some embodiments, the aggregates differentiate into placental organoids during incubation. In certain embodiments, the single-cell suspension is seeded at a density of about 500 cells/spheroid. In some embodiments, the single-cell suspension is centrifuged at about 100×g to 300×g. In some embodiments, the aggregates are incubated in the differentiation media for about 7 days. In other embodiments, the aggregates are incubated in the differentiation media for about 15 days.

In some embodiments, the present invention also features a kit for generating placental organoids. The kit may comprise a medium for culturing a single-cell suspension of naïve-like stem cells and a differentiation medium for inducing the differentiation of the stem cells into trophoblasts. Additionally, in some embodiments, the kit may further comprise instructions for generating the placental organoid, where the instructions describe the steps for culturing the naïve-like stem cells in suspension to generate placental organoids comprising cytotrophoblasts, syncytiotrophoblasts, and extravillous trophoblasts.

One of the unique and inventive technical features of the present invention is the generation of placental organoids in a suspension. Without wishing to limit the invention to any theory or mechanism, it is believed that the technical feature of the present invention advantageously provides for enhanced reproducibility and scalability in organoid development, facilitating more accurate modeling of placental function. None of the presently known prior references or works have the unique inventive technical feature of the present invention.

Moreover, the prior references teach away from the present invention. For example, previous studies have generated trophoblast organoids from either trophoblast stem cells, primary cells, or induced pluripotent stem cells embedded in hydrogels. In contrast, the present invention provides methods for generating placental organoids in suspension without the need for hydrogels. Moreover, methods described herein utilize a single media composition to simultaneously induce differentiation into various trophoblast subtypes, whereas prior approaches required distinct media compositions for stage-specific differentiation.

Furthermore, the inventive technical feature of the present invention not only diverges from prior art but also produces unexpected results that challenge established paradigms in stem cell-derived organoid development. For example, conventional methodologies in organoid generation rely on encapsulation strategies necessitating extracellular matrix (ECM) scaffolds enriched with basement membrane proteins, such as laminin and collagen IV, to induce the polarization and structural organization of cellular populations. The prevailing consensus in prior art dictates that without such biochemical cues, the self-assembly of stem-cell-derived organoids into architecturally functional structures would be infeasible or would result in incomplete differentiation with impaired tissue-like organization. Contrary to this established belief, the present invention demonstrates that self-organizing placental organoids can be derived without exogenous ECM supplementation, thereby defying the foundational assumptions of prior research. This departure from established protocols represents a fundamental shift in understanding how trophoblast lineages and their architectural integrity can be achieved solely through intrinsic self-organization. Unexpectedly, the generated placental organoids exhibit structurally defined cytotrophoblast cores with progressive differentiation into syncytiotrophoblasts and extravillous trophoblasts, mirroring the spatial arrangement and cellular lineage maturation observed in native placental tissue. Furthermore, these organoids undergo autonomous developmental progression in the absence of exogenous differentiation factors, an outcome that contradicts conventional wisdom, which posits that exogenous signaling is necessary to guide trophoblast fate decisions.

In addition, the integration of these placental organoids into microfluidic platforms and drug screening systems has revealed unanticipated functional competencies. Notably, the organoids secrete bioactive factors that modulate angiogenesis and vasculogenesis, akin to native placental tissue, and exhibit a high degree of sensitivity to commonly used pharmaceutical compounds. These findings were unexpected, as prior literature suggests that in vitro trophoblast models often fail to recapitulate the full spectrum of placental secretory functions, particularly in the context of drug-induced responses. The robustness of these functional outputs highlights the invention's potential applications in modeling maternal-fetal interactions, pharmaceutical safety assessment, and placental disease research. The present invention thus contradicts established methodologies, challenges prior assumptions regarding trophoblast differentiation, and provides unexpectedly functional organoid models, positioning it as a significant advancement in the field. By eliminating the necessity for exogenous ECM proteins and differentiation factors, this invention offers a streamlined, cost-effective, and biologically relevant approach to modeling placental development, with implications for regenerative medicine, drug testing, and reproductive health research.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1A, 1B, 1C, 1D, 1E, 1F, and 1G shows the generation and characterization of placenta-like organoids. FIG. 1A shows a schematic representing the generation of placenta-like organoids from naïve and primed stem cells. FIG. 1B shows a brightfield image of morphology of naïve vs. primed stem cells (left) and representative immunofluorescence expression of pluripotency marker SOX2 and naïve pluripotency marker TBX3 (right). FIG. 1C shows qRT-PCR based comparative gene expression analysis of trophoblast markers GATA3 and TFAP2C in differentiated cells obtained in 2D, suspension and Matrigel. FIG. 1D shows immunofluorescence images depicting the expression of trophoblast markers TFAP2C, KRT7, HLA-G, FN1, GDF15, and MMP2 on day 7 and day 15 of differentiation. FIG. 1E shows a positive pregnancy result on a commercial pregnancy test. FIG. 1F shows hCG ELISA representing the hCG concentration obtained by the differentiation methods. FIG. 1G shows GDF15 secretion quantified by GDF15 ELISA in 2D, in suspension and in BME.

FIG. 2A, 2B, 2C, 2D, 2E, 2F, 2G, and 2H show single-cell transcriptome analysis of placenta-like organoids. FIG. 2A shows a schematic demonstrating the experimental procedure of dissociating and labeling single cells generated from placental organoids prior to sequencing. FIG. 2B shows phenotypic UMAP representing the cell clusters obtained from primed stem cells, naïve stem cells, day 7 and day 15 placenta-like organoids. FIG. 2C shows a MAP depicting the unique populations in day 7 and day 15 placenta-like organoids. FIG. 2D shows violin plots indicating the expression of pluripotency genes, trophoblasts markers and mural cells. FIG. 2E shows a stacked bar graph denoting the fraction of cells in placental organoids. FIG. 2F shows dot plots indicating the expression of cytotrophoblasts (CTB), syncytiotrophoblasts (STB) and extravillous trophoblasts (EVT). FIG. 2G shows gene ontology analysis depicting the enriched biological pathways. FIG. 2H. shows dot plots showing key transcriptional factors in trophoblast differentiation.

FIG. 3A, 3B, 3C, 3D, 3E, 3F, 3G, and 3H shows analysis of placental secretome on microvasculature-on-a-chip. FIG. 3A shows a schematic representing the experimental plan of assessing the effect of secretome on angiogenesis and vasculogenesis. FIG. 3B shows the effect of placental secretome on microvascular angiogenic sprouting. FIG. 3C shows the effect of the placental secretome on microvascular vasculogenesis. FIG. 3D shows perfusability of day 7 and day 15 placental organoid conditioned microvasculature. FIG. 3E shows placental secretome analysis using an angiogenesis cytokine array. FIG. 3F. IL-6 and FIG. 3G shows an ELISA demonstrating cytokine release in day 7 and day 15 placental organoid conditioned media. FIG. 3H shows qRT-PCR gene expression of inflammatory markers EDN1, ICAM1, NFE2L2, MMP2, and MMP9.

FIG. 4A, 4B, 4C, 4D, and 4E shows placenta organoids as an in vitro drug testing and implantation platform. FIG. 4A shows a schematic representing the experimental time frame for drug toxicity testing on placental organoids. FIG. 4B shows d7 and d15 placental organoids treated with increasing doses of nicotine, acetaminophen, ibuprofen, lumiracoxib, enabling drug toxicity testing. FIG. 4C shows a schematic representing the experimental time frame for the implantation of placental organoids on Ishikawa endometrial cells. FIG. 4D shows brightfield images representing the Ishikawa cells containing lower chambers of Transwells and migration of CellTracker labeled placental organoids on day one of co-culture. The red migrating cells were quantified on d1 and d2 of co-culture. FIG. 4E shows a schematic representing the steps involved in implantation: apposition, adhesion, and invasion. Brightfield images representing the BME encapsulated Ishikawa cell droplets, and adherence of CellTracker labeled placental organoids on day one of co-culture. The percentage of placental organoids undergoing apposition, adhesion, and invasion with day 7 and day 15 placental organoids were quantified and immunostained with TFAP2C to show migrating cells.

FIG. 5A shows qRT-PCR analysis reveals lower expression of NANOG, OCT4, and higher expression of TBX3 in naïve stem cells.

FIG. 5B shows that naïve stem cells demonstrate a higher proliferation rate as compared to primed stem cells.

FIG. 5C shows placental organoids expressing trophoblast markers TFAP2C and KRT7 are generated in Matrigel, in suspension, and in 2D.

FIG. 5D shows naïve stem cell-derived placental organoids show higher expression of trophoblast markers GATA3 and TFAP2C as compared to primed stem cell-derived placental organoids.

FIG. 5E shows placental organoids were cultured and maintained for extended periods till passage 9.

FIG. 6A shows naïve stem cell-derived placental organoids but not the primed stem cell-derived placental organoids secreted hCG.

FIG. 6B shows naïve stem cell-derived placental organoids showed increased GDF15 secretion on day 15 as indicated by GDF15 ELISA.

FIG. 7A, 7B, and 7C show immunofluorescence staining of trophoblast migration into Ishikawa cells containing BME droplets upon implantation with day 7 and day 15 placental organoids for markers TFAP2C and KRT7 (FIG. 7A), HLA-G and FN1 (FIG. 7B), and GDF15 and MMP2 (FIG. 7C).

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

As used herein, a “suspension culture” refers to a method of culturing cells or cell aggregates in a liquid medium, wherein the cells are maintained in a free-floating state and are not adherent to a solid substrate. In some embodiments, the cells or aggregates within the suspension culture are dispersed within the liquid medium, allowing for homogeneous growth and providing the ability to scale the culture for various applications, including but not limited to, cell expansion, drug screening, and the production of biological products, e.g., organoids.

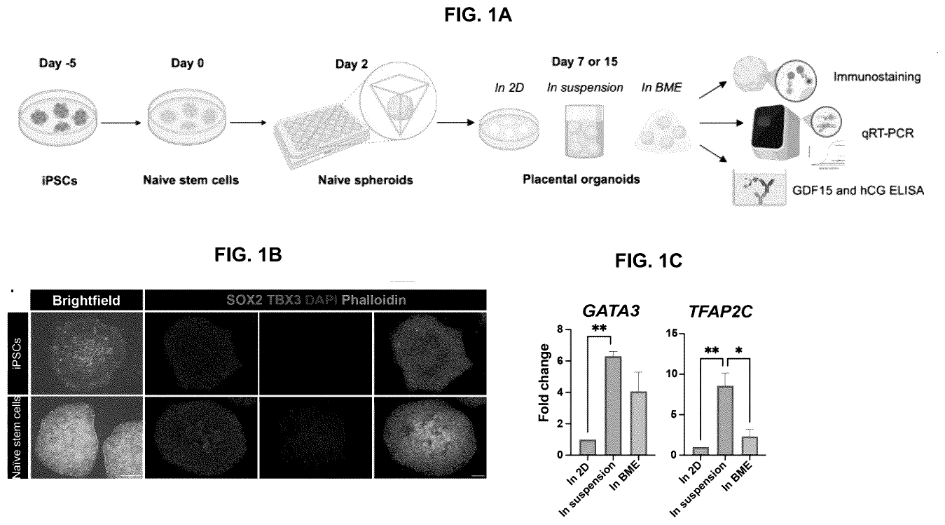

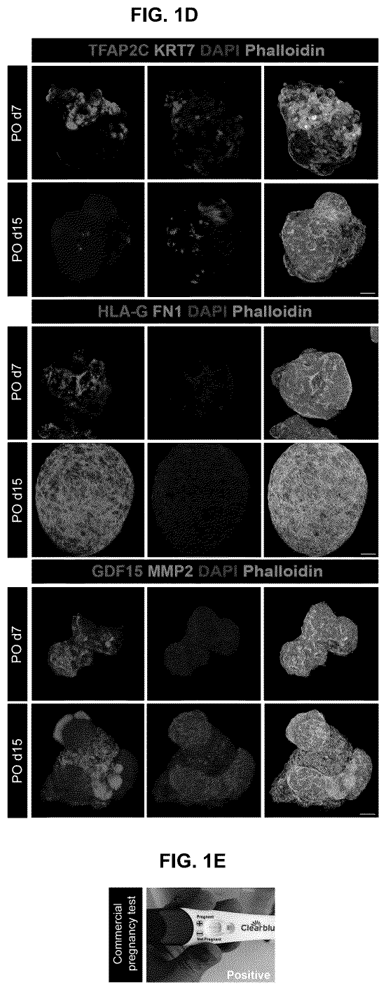

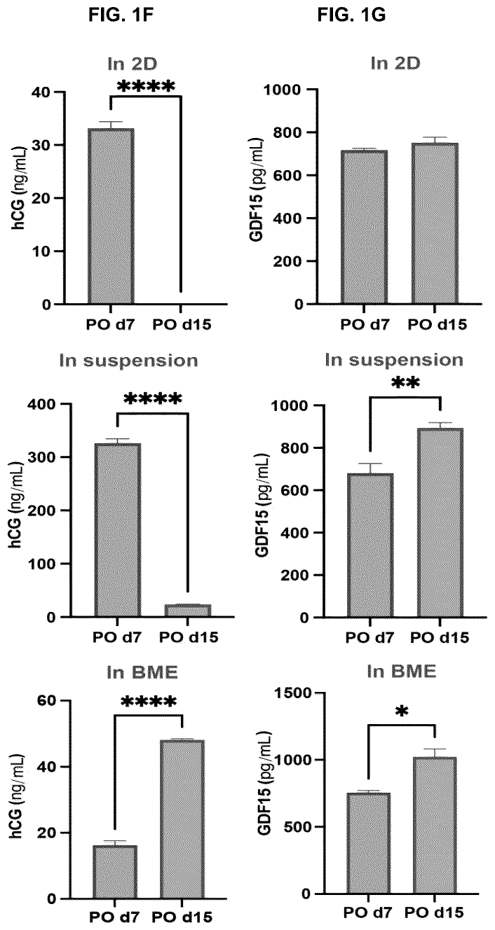

Referring now to FIGS. 1A-7C, the present invention features methods and systems for generating placenta-like organoids in suspension or placenta-on-a-chip systems as well as methods of use.

The present invention encompasses placental organoids that can be used in suspension culture or integrated into placenta-on-a-chip systems. The placental organoids described herein provide a versatile platform for modeling placental function and development in vitro, whether cultured in suspension to mimic organoid formation or incorporated into microfluidic systems for advanced, dynamic studies of placental biology and drug responses. The present invention allows for flexible application in both traditional suspension culture settings and cutting-edge placenta-on-a-chip technologies, expanding the potential for high-throughput screening, toxicity testing, and disease modeling.

The present invention features a placental organoid composition comprising a plurality of trophoblast cells derived from naïve-like stem cells (e.g., RSeT™ stem cells) in a suspension culture. In some embodiments, the organoid exhibits a three-dimensional architecture resembling human placental tissue. In some embodiments, the organoid demonstrates functional properties characteristic of human placental tissue, including expression of human leukocyte antigen-G (HLA-G), and the secretion of pregnancy related hormones. Alternatively, in other embodiments, the organoid demonstrates functional properties characteristic of human placental tissue, including expression of human leukocyte antigen-G (HLA-G), transcription factor AP-2 Gamma (TFAP2C), Keratin 7 (KRT7), or a combination thereof and the secretion of pregnancy related hormones including growth differentiation factor 15 (GDF15), Human Chorionic Gonadotropin (hCG), or a combination thereof.

The present invention is not limited to the expression and secretion of the aforementioned markers, but may also encompass additional markers associated with human placental tissue at various stages of development.

In certain embodiments, the trophoblast cells differentiate into cytotrophoblasts, syncytiotrophoblasts, and extravillous trophoblasts. In some embodiments, the differentiation of syncytiotrophoblasts results in the expression of Syndecan-1 (SDC1), Choriogonadotropin subunit beta (CGB), or a combination thereof. Additionally, in some embodiments, the differentiation of extravillous trophoblasts leads to the expression of HLA-G, growth differentiation factor 15 (GDF15), Matrix Metallopeptidase 2 (MMP2), Fibronectin 1 (FN1), or a combination thereof.

The present invention also includes a method for assessing drug toxicity. In some embodiments, the method comprises administering a drug to the placental organoid composition as described herein for a period of time, followed by measuring the viability of cells within the placental organoid composition.

In some embodiments, cell viability may be evaluated using a luminescence-based assay or other suitable assays. In certain embodiments, cell viability may be assessed using the CellTiter Glo assay®.

In some embodiments, drug toxicity is measured based on the drug dose at which cell survival is reduced to less than 50%. In other embodiments, drug toxicity is measured based on the drug dose at which cell survival is reduced to less than 75%.

In some embodiments, the drug may be nicotine, acetaminophen, ibuprofen, lumiracoxib, or a combination thereof. Additionally, other drugs may be assessed based on factors such as their known or suspected impact on placental function, metabolism, or fetal development. Drug selection may be guided by prior clinical data, toxicology studies, or emerging concerns regarding their effects on pregnancy and placental health.

In some embodiments, the time period is 24 or 48 hours post-administration of a drug. Without wishing to limit the present invention to any theory or mechanism it is believed that these time points are critical for evaluating drug toxicity, as placental cells undergo cell death during this period in response to drug exposure. However, the present invention is not limited to these time points and may also encompass long-term exposure studies to assess a drug's effects on the placental organoids.

The present invention may further comprise a method of screening drugs for toxicity. In some embodiments, the method comprises administering a drug to the placental organoid composition, as described herein, for a period of time and measuring cell viability within the placental organoid composition using a viability assay. In some embodiments, the drug is toxic if the cell viability is reduced below a predetermined threshold. In certain embodiments, the drug is toxic if the cell viability is reduced to less than 50%. In other embodiments, the drug is toxic if the cell viability is reduced to less than 75%.

In some embodiments, the viability assay is a luminescence-based viability assay (e.g., CellTiter Glo assay®).

The aforementioned screening method may be performed manually in a multi-well plate format, such as a 96-well plate, or using automated robotic systems for high-throughput screening. Without wishing to limit the present invention to any theory or mechanism, it is believed that the method described herein allows for flexible adaptation to various scales and platforms, enabling the efficient assessment of drug toxicity across multiple organoid samples.

The present invention may further feature a method of generating a placental organoid composition (i.e., a placental organoid). In some embodiments, the method comprises obtaining a single-cell suspension of naïve-like stem cells, seeding the single-cell suspension onto a culture surface in a first medium, centrifuging the single-cell suspension to facilitate aggregate formation, incubating the single cell suspension to form aggregates, and replacing at least a portion of the first media with a differentiation media and incubating the aggregates for a period of time. In some embodiments, the aggregates differentiate into placental organoids during incubation. In certain embodiments, the single-cell suspension is seeded at a density of about 500 cells/spheroid. In some embodiments, the single-cell suspension is centrifuged at about 100×g to 300×g. In some embodiments, the aggregates are incubated in the differentiation media for about 7 days. In other embodiments, the aggregates are incubated in the differentiation media for about 15 days.

The present invention may feature a method of generating a placental organoid composition (i.e., a placental organoid). In some embodiments, the method comprises washing naïve-like stem cells in a cell compatible buffer (e.g., PBS), and dissociating the naïve-like stem cells to obtain a single cell suspension (e.g., a single-cell suspension of naïve-like stem cells). The single cell suspension may then be seeded onto a culture surface (e.g., a treated plate; e.g., an anti-adherence rinsing solution treated plate) in a first medium. In some embodiments, the first medium is supplemented with a ROCK inhibitor, e.g., Y-27632 dihydrochloride. Next, in some embodiments, the single cell suspension may be centrifuged to facilitate aggregate formation. After centrifugation, the single-cell suspension may be incubated for a period of time to allow the formation of aggregates from the single cells within the suspension. In some embodiments, the method further comprises replacing at least a portion (e.g., about 70-80%) of the first media with a second media (e.g., a differentiation media) and incubating the aggregates in the second media for a period of time. In some embodiments, the aggregates are maintained in the second media (e.g., the differentiation media, such as TSC differentiation medium) for a period of time (e.g., 7 or 15 days). In some embodiments, Early-stage placental organoids are generated by day 7, while late-stage placental organoids are generated by day 15. Maintaining the aggregates in the differentiation media (e.g., TSC medium) induces differentiation from naïve stem cells to trophoblasts. Among the trophoblast types, early stage placental organoids have more cytotrophoblasts and syncytiotrophoblasts while the later stage organoids have more extravillous trophoblasts.

In some embodiments, the single cell suspension is seeded at a seeding density of about 500 cells/spheroid. In some embodiments, the single cell suspension is seeded at a seeding density of about 600 cells/spheroid. In some embodiments, the single cell suspension is seeded at a seeding density of about 400 cells/spheroid. In some embodiments, the single cell suspension is seeded at a seeding density of about 400-600 cells/spheroid. Without wishing to limit the present invention to any theory or mechanism, it is believed that specific cell density at which the single-cell suspension is seeded corresponds to the resultant size of the placental organoid. The starting cell number is also important for efficient differentiation and reproducibility.

In some embodiments, the single cell suspension is centrifuged at about 300×g. In some embodiments, the single cell suspension is centrifuged at about 200×g. In some embodiments, the single cell suspension is centrifuged at about 100×g. In some embodiments, the single cell suspension is centrifuged at about 100×g to 300×g. Again, without wishing to limit the present invention to any theory or mechanism it is believed that centrifuging the single-cell suspension at higher speeds may result in cell death, while lower speeds may fail to facilitate aggregation. In certain embodiments, a speed of 300×g promotes tighter aggregate formation.

In some embodiments, the single cell suspension is centrifuged at about 5 minutes. In some embodiments, the single cell suspension is centrifuged at about 10 minutes. In some embodiments, the single cell suspension is centrifuged at about 2.5 minutes.

In some embodiments, the single cell suspension is incubated for one day to allow for aggregate formation. In some embodiments, the single cell suspension is incubated for two days to allow for aggregate formation. In some embodiments, the single cell suspension is incubated for three days to allow for aggregate formation. In some embodiments, the single cell suspension is incubated for five days to allow for aggregate formation.

In some embodiments, about 60% of the first media is replaced with the second media. In some embodiments, about 70% of the first media is replaced with the second media. In some embodiments, about 80% of the first media is replaced with the second media. In some embodiments, about 90% of the first media is replaced with the second media. In some embodiments, about 99% of the first media is replaced with the second media.

In some embodiments, the second media (e.g., the differentiation media) comprises a tissue culture media (e.g., Dulbecco's Modified Eagle's Medium/Nutrient Ham's Mixture F-12 (DMEM/F-12)). In some embodiments, the tissue culture media may be supplemented with either N2, B-27 or a combination of both. Additionally, in some embodiments, the tissue culture media may be supplemented with growth factors, including but not limited to epidermal growth factor (EGF), hepatocyte growth factor (HGF), fibroblast growth factor 2 (FGF-2). In some embodiments, the tissue culture media may be supplemented with proteins including but not limited to R-Spondin-1, Prostaglandin E2 (PGE2), or a combination thereof. In other embodiments, the tissue culture media may be supplemented with kinase inhibitors including but not limited to CHIR 99021, A83-01, SB431542, Y-27632, or a combination thereof. The present invention is not limited to the aforementioned factors for supplementing the tissue culture medium and may include any additional suitable factors as appropriate.

In some embodiments, the present invention also features a kit for generating placental organoids. The kit may comprise a medium for culturing a single-cell suspension of naïve-like stem cells and a differentiation medium for inducing the differentiation of the stem cells into trophoblasts. Additionally, in some embodiments, the kit may further comprise instructions for generating the placental organoid, where the instructions describe the steps for culturing the naïve-like stem cells in suspension to generate placental organoids comprising cytotrophoblasts, syncytiotrophoblasts, and extravillous trophoblasts.

In some embodiments, the differentiation medium comprises a tissue culture medium, such as Dulbecco's Modified Eagle's Medium/Nutrient Ham's Mixture F-12 (DMEM/F-12). The tissue culture medium may be supplemented with N2, B-27, or a combination thereof. Additionally, in some embodiments, the medium may be supplemented with one or more growth factors, including but not limited to epidermal growth factor (EGF), hepatocyte growth factor (HGF), and fibroblast growth factor 2 (FGF-2). The medium may also include proteins such as R-Spondin-1, Prostaglandin E2 (PGE2), or a combination thereof. In certain embodiments, kinase inhibitors may be added, including but not limited to CHIR 99021, A83-01, SB431542, Y-27632, or combinations thereof. The present invention is not limited to these specific supplements and may include any additional suitable factors as appropriate.

### EXAMPLE

The following is a non-limiting example of the present invention. It is to be understood that said example is not intended to limit the present invention in any way. Equivalents or substitutes are within the scope of the present invention.

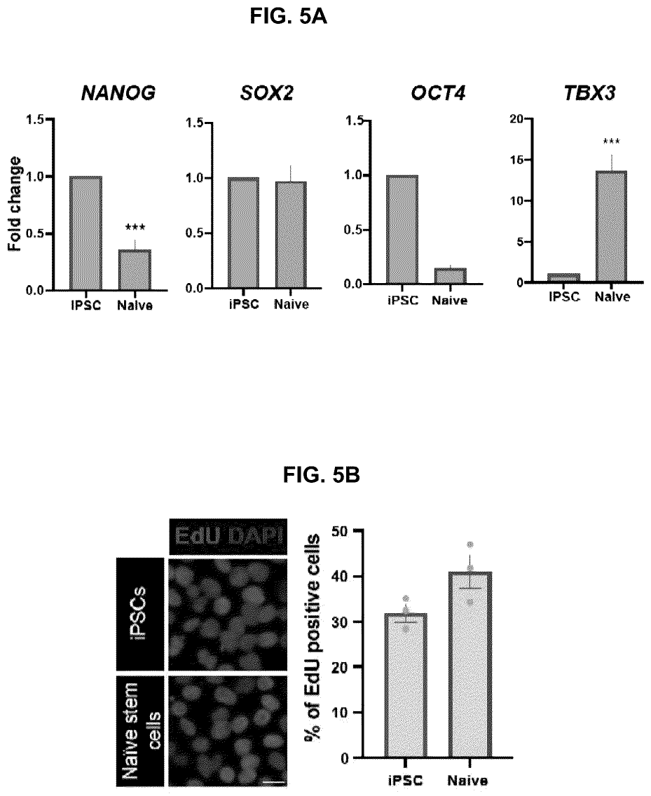

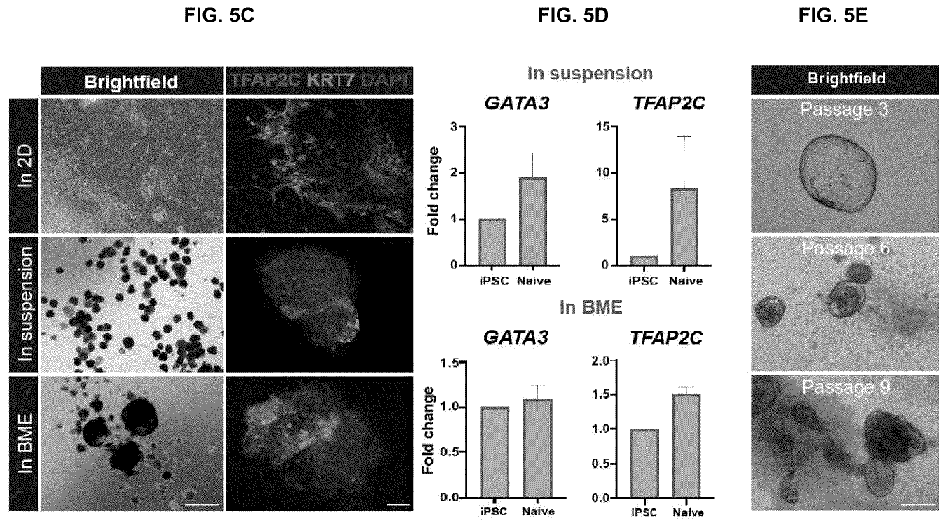

iPSC-derived placental organoids mimic in vivo trophoblast markers and function. To directly compare the differences in naïve vs. primed stem cell-derived placental organoids, both naïve stem cells and primed iPSCs were differentiated into placental organoids using the same differentiation protocol as described in the schematic (FIG. 1A). Naïve stem cells generated from primed iPSCs demonstrated dome-like colony morphology with sharp edges (FIG. 1B), expressed pluripotency markers SOX2, NANOG, OCT4 and increased levels of TBX3 (FIG. 5A). Furthermore, naïve stem cells demonstrated higher proliferation rate as compared to primed stem cells as indicated by EdU assay (FIG. 5B). Various methods have been described for the generation of human trophoblast cells from both naïve and primed stem cells. But these studies have not provided a comprehensive comparison of the various differentiation protocols. Hence, to investigate the efficiency of these methods, placental organoids were generated by 3D differentiation in BME drops, 3D differentiation in suspension, and 2D trophoblast differentiation (FIG. 5C). It was also observed that spheroid formation enhances placental organoid generation. Upon 2D differentiation of naïve stem cells into TSCs, the cells did not aggregate in Aggrewell plates, demonstrating migrating cells that adhered to the plate surface and prevented aggregation. Therefore, these experiments suggested that although the same media conditions were used for the differentiation, the initial structural morphology determines the subsequent expression of characteristic markers. Recent studies have controversially demonstrated that human naïve stem cells, but not primed stem cells are able to differentiate into trophoblast stem cells. Using the same differentiation protocol, naïve stem cells led to higher expression of GATA3 and TFAP2C as compared to primed stem cells under both culture conditions, in BME and in suspension (FIG. 1C). Specifically, the suspension method for differentiating stem cells into trophoblasts led to improved trophoblast expression as demonstrated by KRT7 and TFAP2C immunofluorescence (FIG. 5C and 5D). To assess the stability of these cultures in BME, the placental organoids were maintained in culture for up to 10 passages (FIG. 5E). Along the passages it was observed that the migrating EVT-like cells were found to increase over a period of time with the maturation of the placenta-like organoids.

Naïve stem cell-derived day 7 and day 15 placental organoids generated in suspension were characterized by immunostaining with KRT7, TFAP2C, HLA-G, GDF15, MMP2, and FN1 (FIG. 1D). The expression cytotrophoblast marker KRT7 was found to be increased in day 7 placental organoids. The stage-specific syncytiotrophoblast markers such as TFAP2C and GDF15 were highly expressed in day 15 placental organoids and day 7 placental organoids, respectively. Extravillous trophoblast marker HLA-G and MMP2 expression were found to be increased in day 15 placental organoids. However, FN1 deposition was higher in day 15 placental organoids.

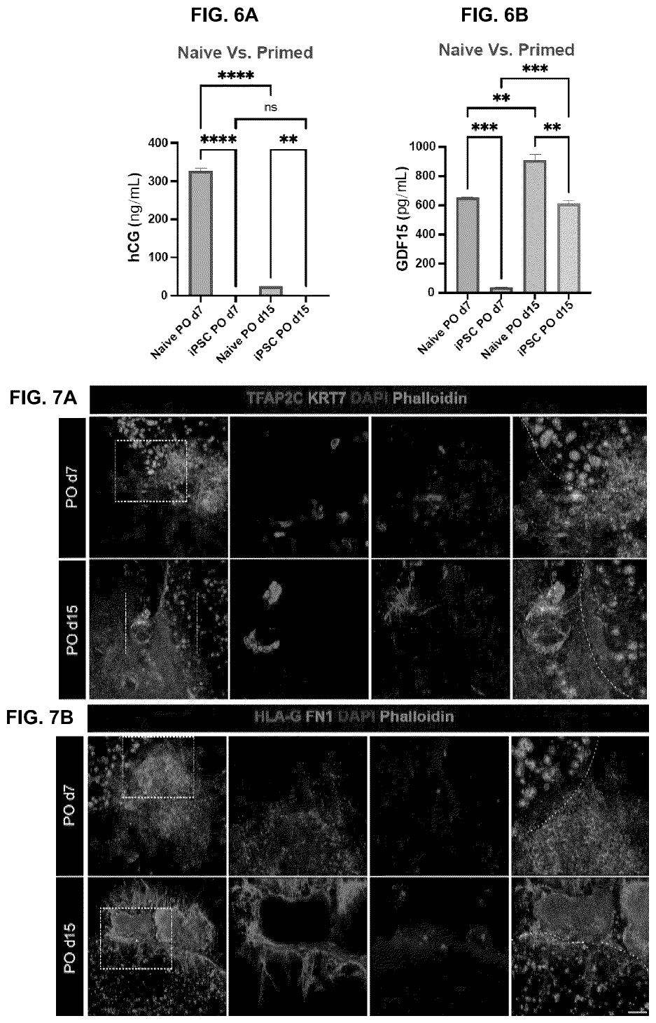

hCG is a predominant hormone produced by the villous syncytiotrophoblasts during pregnancy indicative of endometrial receptivity. The placental organoid secretome confirmed a ‘pregnant’ phenotype with an over-the-counter pregnancy test suggesting hCG secretion (FIG. 1E). To further detect the hCG levels in naïve vs. primed iPSCs differentiated using the same approach, the secretome of day 7 and day 15 placental organoids was assessed by ELISA (FIG. 6A). While both naïve stem cell-derived day 7 and day 15 placental organoids secreted hCG, it was observed that on day 7 the hCG levels were found to be increased to 326.43±8.02 ng/ml. However, the primed iPSC derived day 7 and day 15 placental organoids were unable to secrete hCG in suspension, thereby questioning the functional ability of these placental organoids. However, recent studies have shown that primed iPSCs differentiation required a modified protocol involving a BMP4 pulse directly generated functional trophoblasts. Hence, given that identical placental organoid differentiation protocol was used for both naïve and primed iPSCs, primed iPSCs did not produce hCG. Further, to assess the effect of culture condition on hCG secretion, ELISA showed that the suspension culture led to higher hCG, particularly on day 7 (FIG. 1F), suggesting the presence of higher numbers of hCG-producing syncytiotrophoblasts. Interestingly, the placental organoids embedded in BME underwent slightly different developmental processes demonstrating lower hCG on day 7 with 16.29±1.29 ng/ml as compared to day 15, 48.18±0.3 ng/ml. 2D-based differentiation, however, led to decreased levels of hCG 33.19±1.2 ng/ml on day 7 to no detectable hCG secretion on day 15.

Growth differentiation factor 15 (GDF15) is a member of the transforming growth factor-β (TGF-β) superfamily, involved in the recruitment and activation of SMAD family of transcription factors that control gene expression. Although GDF15 is weakly expressed in most tissues, it is highly expressed in placental cells during pregnancy and elevated levels of GDF15 are circulated. GDF15 is primarily secreted by the extravillous trophoblasts and decidual stromal cells in the placenta. Therefore, the GDF15 levels secreted by day 7 and day 15 placental organoids into conditioned media were detected by GDF15 ELISA (FIG. 1G). Naïve stem cell-derived placental organoids secreted higher GDF15 as compared to primed iPSC-derived placenta organoids (FIG. 6B). The GDF15 secretion in 2D did not vary depending on the differentiation stage. However, in suspension cultures, day 15 placental organoids demonstrated higher levels of GDF15, corresponding to 894.4±24.9 ng/ml as compared to day 7 placental organoids, 680.9±45.68 ng/ml. Similarly, BME embedded placental organoids secreted higher GDF15 on day 15, 1024.0±57.34 and 757.4±13.93 ng/mL on day 7. Increased GDF15 levels on day 15 placental organoids correlate to higher number of EVT cells on day 15 of differentiation.

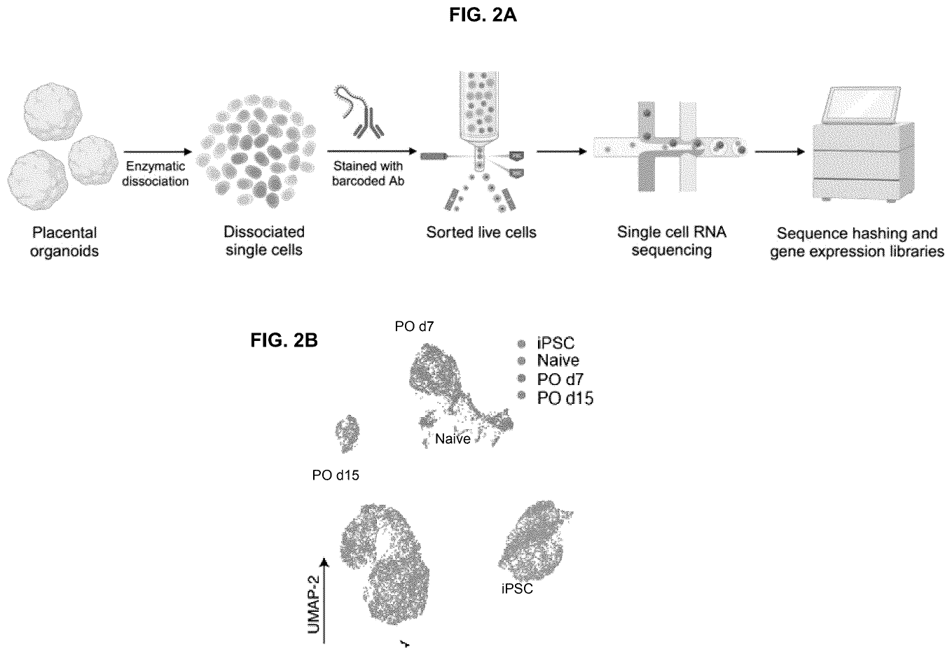

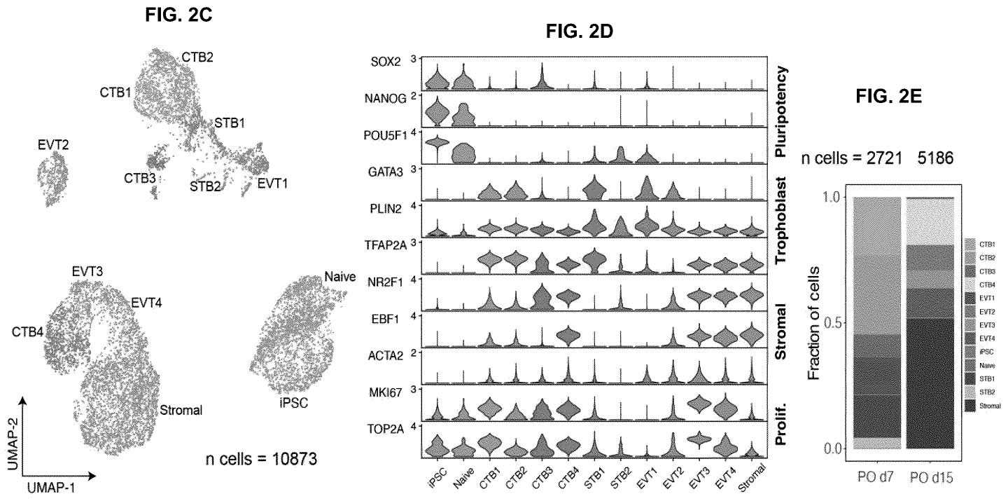

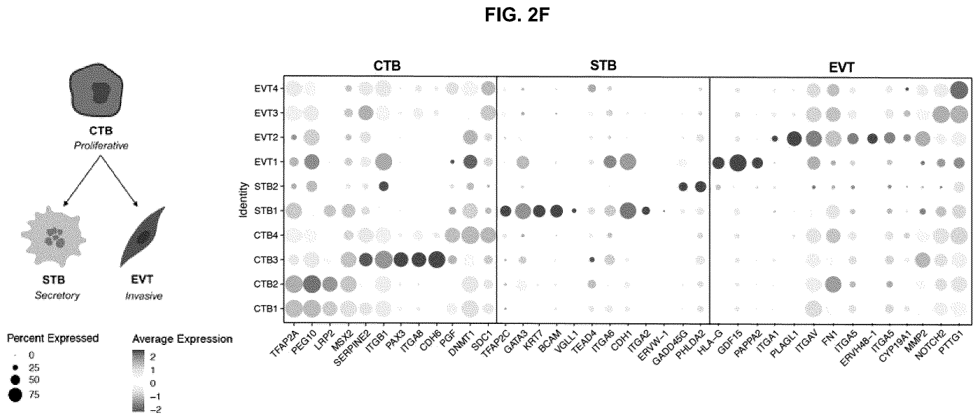

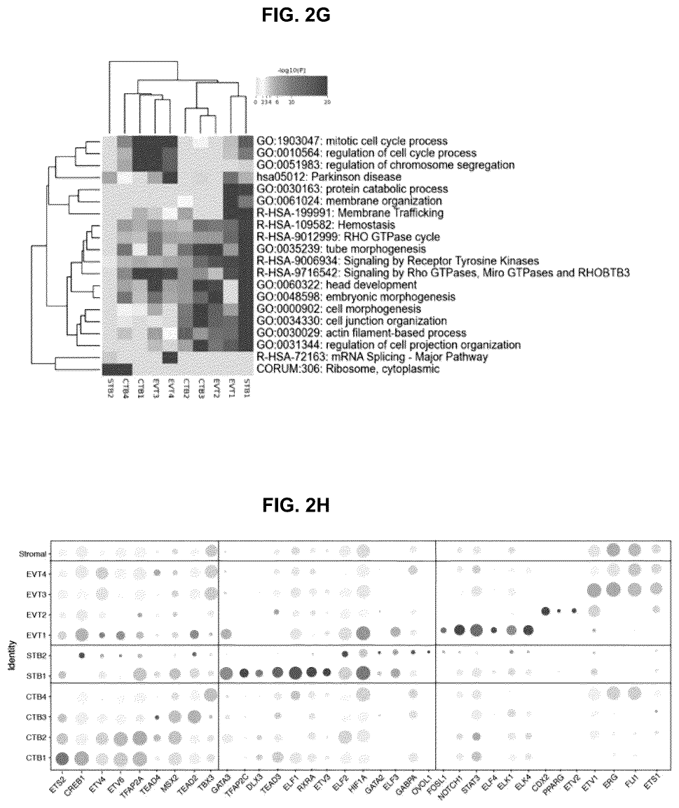

Single cell transcriptomes reveal complexity, cellular heterogeneity and resemblance to in vivo signatures. The developmental trajectory during the differentiation of iPSCs to placental organoids was traced using stage-specific placental organoids, including iPSC spheroids, naïve stem cell spheroids, day 7 placental organoids and day 15 placental organoids. The cellular heterogeneity and composition were determined by dissociation of the organoids into a single cell suspension and generation of cDNA libraries for single-cell RNA sequencing using the 10× Genomics platform, as shown in the schematic (FIG. 2A).

Phenotype based uniform manifold approximation and projection (UMAP) demonstrated the mutually exclusive cell clusters representing the different samples including iPSCs, naïve stem cells, day 7 placental organoids and day 15 placental organoids (FIG. 2B). Dimensional reduction analysis using UMAP showed that the cells from the samples iPSCs, naïve stem cells, day 7 placental organoids and day 15 placental organoids clustered into 13 different clusters (FIG. 2C). The iPSC and naïve stem cell clusters were marked by the elevated expression of pluripotency markers such as SOX2, NANOG and POU5F1 (FIG. 2D). The naïve stem cells derived from primed iPSCs however were embedded in two different regions, one cluster involving the iPSC population which could indicate the presence of pluripotency or undifferentiated stem cells and the other cluster around the STB trophoblast cells. These clusters expressed the naive markers including TBX3, KLF4, GATA6 and ZFP42. The trophoblast cells expressing GATA2, GATA3, KRT7, TFAP2A, and TFAP2C, obtained from day 7 and day 15 placental organoids clustered separately in the UMAP indicating varying levels of trophoblast maturity and cellular transition states. Proliferating cells were marked by the expression of previously published proliferation markers including MKI67 and TOP2A. The fraction of CTB clusters were found to be highest on day 7 while day 15 placental organoids comprised of a large portion of stromal cells (FIG. 2E).

The placental organoids on day 7 and day 15 of differentiation comprised of discrete trophoblast transitional stages: four CTB, two STB and four EVT clusters (FIG. 2F). The CTB1 and CTB2 clusters expressed TFAP2A, PEG10, LRP2, LIN28B, PARP1, TEAD4 and MSX2. The CTB3 cluster demonstrated higher expression of FBN2, SERPINE2, ITGB1, C4orf48, PAX3, ITGA8 and CDH6. PGF, DNMT1 and SDC1 were expressed in the CTB4 cluster. The STB clusters, STB1 exhibited high expression of TFAP2C and GATA3 which are pan trophoblast markers. Furthermore, these cell clusters were PERP, ERVW-1, ERVFRD-1, GCM1 and KRT7 positive. STB2 is identified as a distinct cell state with higher expression of PHLDA2, SGK1, ARRDC3 and HSPA markers. The cell clusters with high expression of HLA-G and GDF15, which are marker genes identifying EVTs. Among the EVTs, EVT1 expressed HLA-G, GDF15, PAPPA2, PRKAG2, EVT2 expressed higher extracellular matrix components such as ITGA1, ITGAV, FN1, ITGA5 and other markers such as ERVH48-1, PLAGL1, CYP19A1. EVT3 and EVT4 expressed SDC1, NOTCH2, HMGB2, PTTG1 and ADAMTS20. The day 15 placental organoids also consisted of clusters expressing fibroblast-like or stromal markers including EBF1, NR2F1, COL1A1, ACTA2 and ID4.

Gene ontology analysis showed enrichment of GO terms associated with biological processes involving cell morphogenesis, embryonic development, tissue morphogenesis, membrane trafficking, membrane organization and cell cycle regulation (FIG. 2G). The CTB subpopulations were distinct, and the GO terms corroborated that CTB3 and CTB4 were more proliferative indicating self-renewal and associated with cell morphogenesis, embryonic morphogenesis, tube morphogenesis and extracellular matrix (ECM) organization. Given that STBs are secretory cells, GO analysis on STB populations showed upregulation of pathways involved in growth factor responses, cell morphogenesis, cell junctional organization and cell polarity in STB2 as compared to STB1. Rho GTPase signalling is essential for migration and invasion, which are characteristics of EVT cells.

The transcriptional factors (TFs) driving placental development beginning from implantation to trophoblast invasion are not completely understood due to the restricted access to human tissues. However, genetic analysis of explanted human tissues have provided insights into the critical TFs regulating development in the first trimester. Among the trophoblasts, CTBs are the progenitor populations for both STBs and EVTs and for maintaining a self-renewing pool of trophoblast stem cells. Therefore, the TFs associated with CTB maintenance and differentiation such as ETS2, MSX2 are important (FIG. 2H). The trophoectoderm transcription factors GATA3, TEAD4 and TFAP2A continue to express in CTB. The expression of ESSRB, EOMES and SOX2 are found to be lower in human CTBs, although they have been shown to be indispensable to mouse placental development and stem cell maintenance. CREB1 is a primary regulator of CTB fusion resulting in STB cells, thus explaining higher CREB1 expression in CTB1, CTB2 and CTB3 subpopulation cells. OVOL1 is known to promote differentiation by repressing the CTB progenitor state, in agreement with the data that demonstrates increased expression of OVOL1 in STB2 and EVT1 populations. Reports have suggested that DLX3-binding sites are associated with syncytiotrophoblast function, including CSH1, CGA, and HSD3B1, indicating its role in the biochemical differentiation of villous cytotrophoblast cells. PPARG is localized to STB and EVT as confirmed by expression in STB1 and EVT2. Similarly, RXRA and PPARG together promote CTB differentiation to STB and prevent EVT invasion, thereby demonstrating expression in only EVT2. FOSL1, NOTCH1 and STAT3 implicated in the cell motility and invasiveness demonstrate higher expression in EVT populations indicating invasive EVT characteristics. The expression of ETS2 increased in EVT3 and EVT4 subpopulations. Interestingly, ETS2 is also involved in regulation of urokinase-type plasminogen activator (uPA) and metalloproteinases such as MMP9 implicated in invasive implantation. Therefore, the stage-specific TFs coordinate and regulate their emergence during different stages of development and specialization.

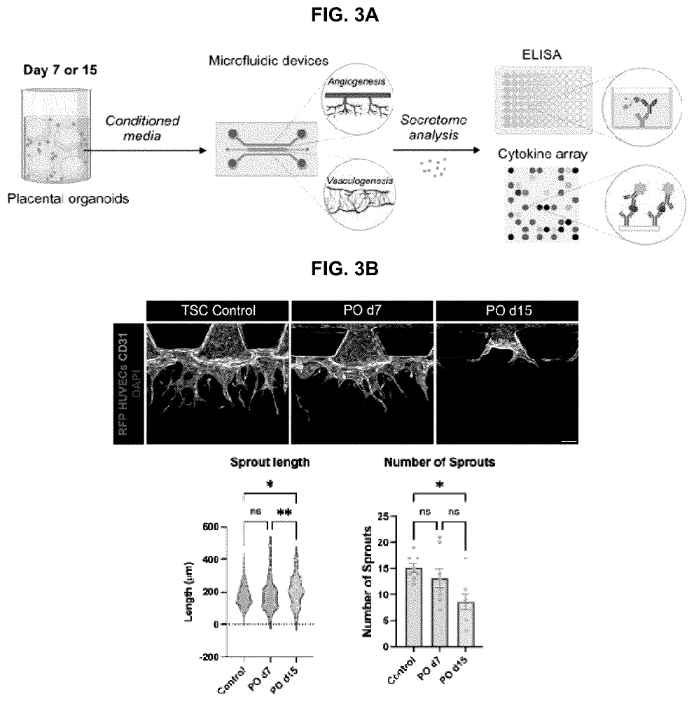

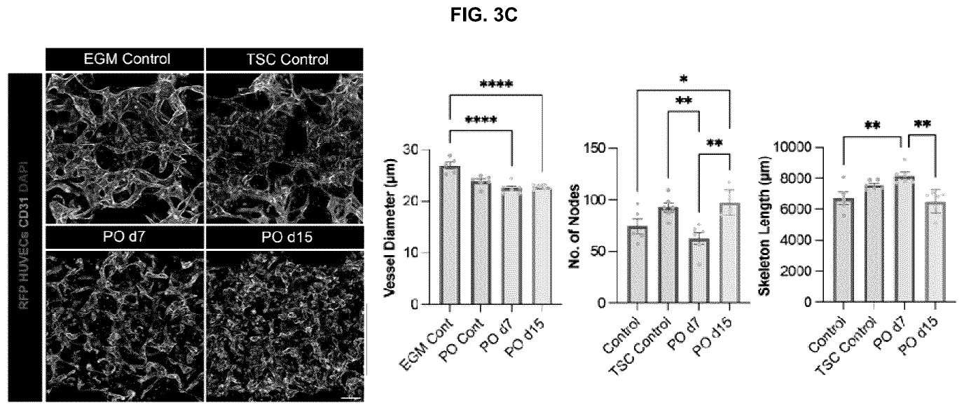

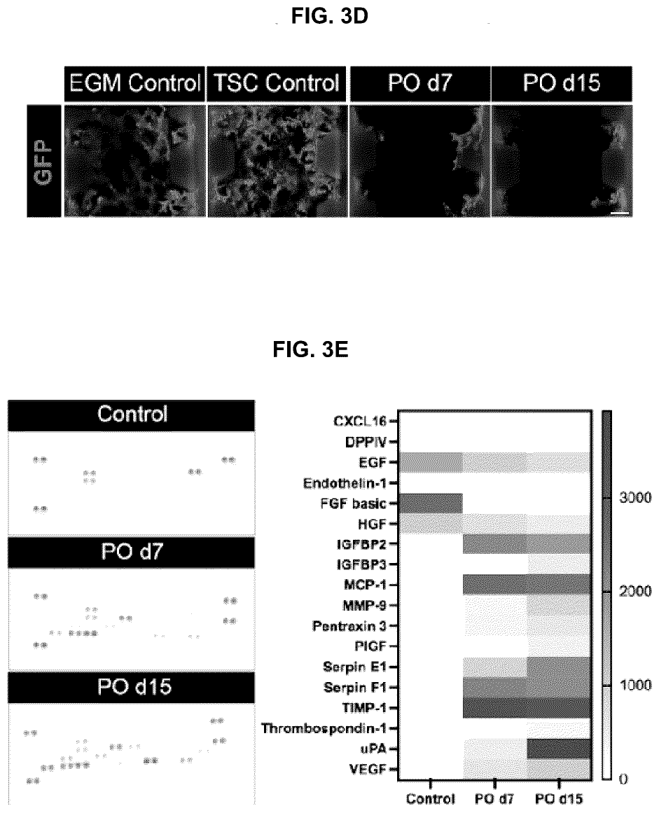

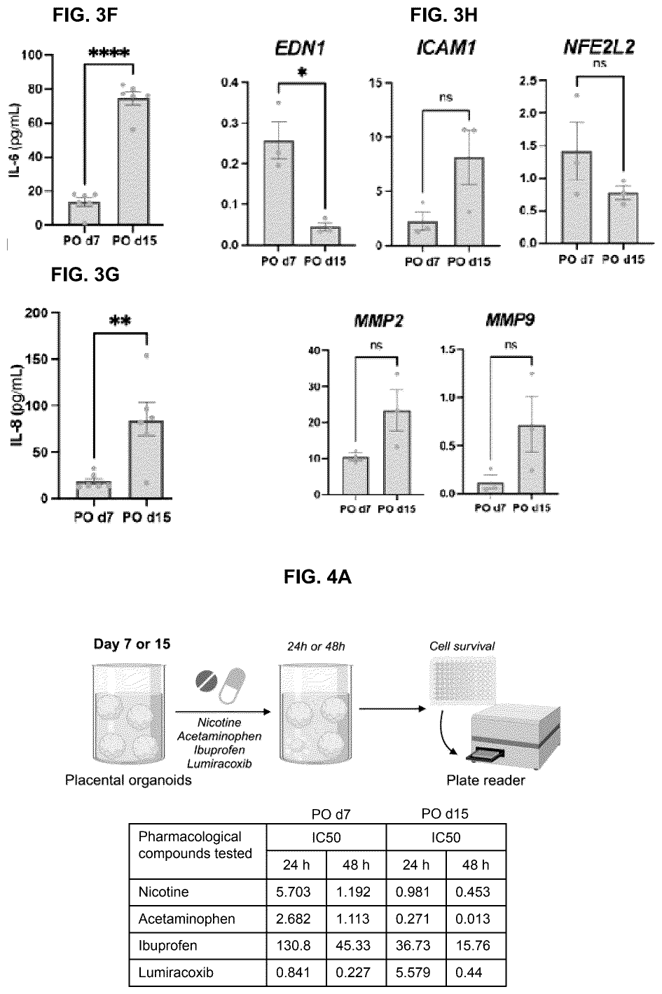

Placental organoids secrete angiogenic factors to modulate angiogenesis and vasculogenesis. Implementing the microfluidics devices, the effect of placental organoid conditioned media on angiogenesis was investigated. The EGM2 endothelial growth media and the TSC placental media was considered as a control. The effect of these media conditions on the angiogenic sprouting of HUVECs was studied in microfluidic devices as demonstrated in the schematic (FIG. 3A). In the control media, HUVECs formed long sprouts. However, shorter sprouts were seen in day 7 and day 15 placental organoid conditioned media conditions (FIG. 3B). The number of sprouts were decreased in placental organoid conditioned media conditions, particularly day 15, yet the sprout length remained similar. In addition, open luminal structures were demonstrated using orthogonal cross-section.

In order to detect the vasculogenic capability of HUVECs under these conditioned media conditions, the self-assembly of vascular networks in microfluidic devices in the presence of placental organoid conditioned media was investigated. It was observed that under both the control conditions, interconnected vascular networks were formed (FIG. 3C). However, the TSC media condition resulted in a thinner increased number of vessels. While the day 7 placental organoid conditioned media led to self-assembled connected vessels, they did not form perfusable networks. Only vacuoles and sprouts were formed in the day 15 placental organoid conditioned media condition demonstrating impaired vascular assembly. Quantification of vascular assembly demonstrated that the vessel diameter did not change between day 7 and 15 placental organoid conditioned media. However, using day 15 placental organoid conditioned media, the number of nodes was increased, and skeleton length decreased. Further, to validate the perfusability of these vascular networks, 1 mm GFP-labelled microbeads were passed through the connected vascular networks. Corroborating with the immunostaining results, the microbeads did not pass through the day 7 and day 15 placental organoid conditioned media conditions (FIG. 3D). Therefore, although interconnected, the vascular networks formed in the presence of placenta conditioned media were not perfusable, implicating the potential role of secretory factors in placental organoid conditioned media.

It is well-established that the highly regulated and coordinated maternal-fetal interactions are facilitated by a plethora of factors, including growth factors, hormones, chemokines and cytokines, secreted either by the maternal cells or the fetal counterparts into the media. Given the influence of placental organoid conditioned media on angiogenesis and vasculogenesis, secretome analysis of the day 7 placental organoids using the angiogenesis cytokine array demonstrated the increase in Endothelin-1, Insulin-like growth factor binding protein-2 (IGFBP2), monocyte chemotactic protein (MCP-1), Serpin family F member 1 (Serpin F1) and Tissue inhibitor of metalloproteinase 1 (TIMP-1) as compared to the day 15 (FIG. 3E). Endothelin-1 is known pro-angiogenic factor through its direct effects on endothelial and peri-vascular cells and indirect action on increasing the release of VEGF. IGFBP2 also promotes angiogenesis by enhancing VEGF gene promoter activity. Another angiogenic chemokine MCP-1 induces TGF-b-induced angiogenesis through its actions on smooth muscle cells. Reports suggest that Serpin F1 inhibits angiogenesis and thereby prevents perfusable vessel formation. TIMP-1 is known to block endothelial response to angiogenic factors, MMP activity, and endothelial tube formation. While the pro-angiogenic factors Endothelin-1, IGFBP2 and MCP-1 promote angiogenic sprouting and vessel connections, the anti-angiogenic factors Serpin F1 and TIMP-1 prevent the formation of perfusable vascular networks in the presence of day 7 placental organoid conditioned media. However, day 15 placental organoids secreted higher levels of CXC motif chemokine ligand 16 (CXCL16), insulin-like growth factor binding protein-3 (IGFBP3), matrix metalloproteinase 9 (MMP-9), pentraxin 3 (PTX3), placenta growth factor (PIGF), serpin E1, uPA and vascular endothelial growth factor (VEGF). Among these factors, CXCL16 is a pro-angiogenic cytokine promoting endothelial cell proliferation, migration and vessel formation in vitro. Both pro- and anti-angiogenic roles of IGFBP3 have been reported. On one hand, IGFBP3 promotes angiogenic sprouting and maintenance of tip phenotype, while on the other hand, it inhibits angiogenesis and tube formation. Secreted MMP9 promotes angiogenesis through the release of a potent angiogenic factor VEGF, which was also found to be increased in the day 15 placental organoid secretome. Pentraxin 3 is a critical mediator of angiogenesis and is known to both promote and inhibit angiogenic processes depending on interacting factors. PIGF is a member of the VEGF family, known to strongly stimulate angiogenesis through its direct and indirect activation of VEGFR-1. Similarly, serpin E1 and uPA promote angiogenesis and mediate vascular permeability. Angiogenesis is regulated by the critical balance between pro-and anti-angiogenic factors as well as the interactions with the extracellular matrix components. Therefore, given the pro-and anti-angiogenic effects of these factors, the cumulative effect determines vascular assembly and vessel formation of endothelial cells in microfluidic devices.

Among the cytokines, the placental cells CTB, STB, EVT, decidual stromal cells, and certain immune cells express interleukin-6 (IL-6). IL-6 production was found to be increased with gestational age. In line with these findings, higher IL-6 levels of 74.68±3.8 pg/mL were detected in the conditioned media of placental organoids on day 15 as compared to day 7 (FIG. 3F). IL-8 however, was reported to be expressed by decidual stromal cells, glandular epithelium and specific immune-related cells in the placenta. Findings from first and second trimester placenta explants also showed that IL-8 significantly increases during the second trimester and at term. Comparably, the secretion of IL-8 by placental organoids increased from d7 (18.44±3.38 pg/mL) to d15 (83.98±18.06 pg/mL) of placental differentiation (FIG. 3G). In agreement with the secretome findings, qRT-PCR showed that while EDN1, NFE2L2 was increased in day 7 placental organoids, the expression of ICAM1, MMP2 and MMP9 was increased in day 15 placental organoids (FIG. 3H). The higher expression of pro-angiogenic EDN1 and NFE2L2 in day 7 placental organoids further corresponds to the enhanced angiogenesis observed in microfluidic devices upon exposure to day 7 placental organoid conditioned media. Although ICAM1 and MMP2 are known to modulate angiogenesis, MMP9 may block angiogenesis through the generation of angiostatin.

Placental organoids provide a platform for in vitro drug testing. It is well-established that ethical constraints hamper the testing of drugs on pregnant women. Furthermore, maternal drug usage is known to promote a range of conditions, such as fetal drug tolerance, antibiotic resistance, toxicity, obesity and diabetes. While it is rendered safe to use certain drugs during pregnancy, the actual short as well as long-term effects of these drugs on fetal health cannot be currently studied. Therefore, as an application of the placental organoids systems in drug toxicity testing, these organoids were treated with commonly used compounds such as nicotine, acetaminophen, ibuprofen and lumiracoxib. Nicotine is a highly studied compound known to easily cross the placental barrier and reach a fetal concentration of 15% higher than mother. Reports suggest that approximately 5-8% pre-term deliveries, 13-19% of low-birthweight term infants, 22-34% cases of sudden infant deaths and 5-7% of pre-term deaths are linked to prenatal maternal smoking. Hence, considered toxic to the developing fetus either directly or indirectly. Acetaminophen, also known as paracetamol, is usually considered as a safe antipyretic and analgesic medicine for pregnant women. However, acetaminophen and its metabolites easily cross the placenta and are potentially toxic as it has previously been detected in the fetus and is implicated in adverse birth and later life health outcomes. Nonsteroidal anti-inflammatory drugs (NSAIDs) such as Ibuprofen are the most commonly used medications for the treatment of pain, inflammation and fever during pregnancy. Ibuprofen readily enters fetal circulation, raising concerns about its long-term effects on fetal health and development. While the use of another NSAID, Lumiracoxib, has not been adequately investigated. Furthermore, despite the popularity worldwide, these highly selective COX-2 inhibitors have been withdrawn commercially owing to the serious adverse effects. Therefore, it has been recommended that it should not be used during the first two trimesters of pregnancy unless it justifies the potential risk to the fetus. Day 7and day 15 placental organoids were treated with increasing doses of drugs such as nicotine, acetaminophen, ibuprofen and lumiracoxib. The drug toxicity and IC₅₀ value of the drugs was determined based on cell survival at 24 and 48 hours post drug exposure. At the 24 hour time point, the day 7 placental organoids displayed IC₅₀ 5.70 mg/mL, 2.68 mM, 130.8 mg/mL and 0.841 mM for nicotine, acetaminophen, ibuprofen and lumiracoxib, respectively. However, at the 48 hour time point the IC₅₀ dropped to 1.19 mg/mL, 1.11 mM, 45.33 mg/mL and 0.22 mM. The day 15 placental organoids on the other hand, showed higher sensitivity to the drugs with lower IC₅₀ values. Therefore, these day 7 and day 15 placental organoids could reproducibly be used for drug toxicity screening in vitro.

Placental organoids model human implantation in vitro

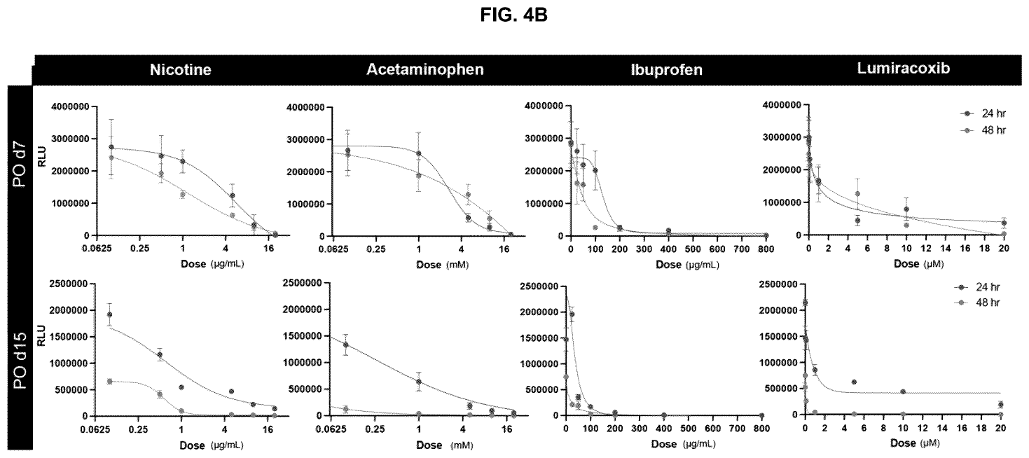

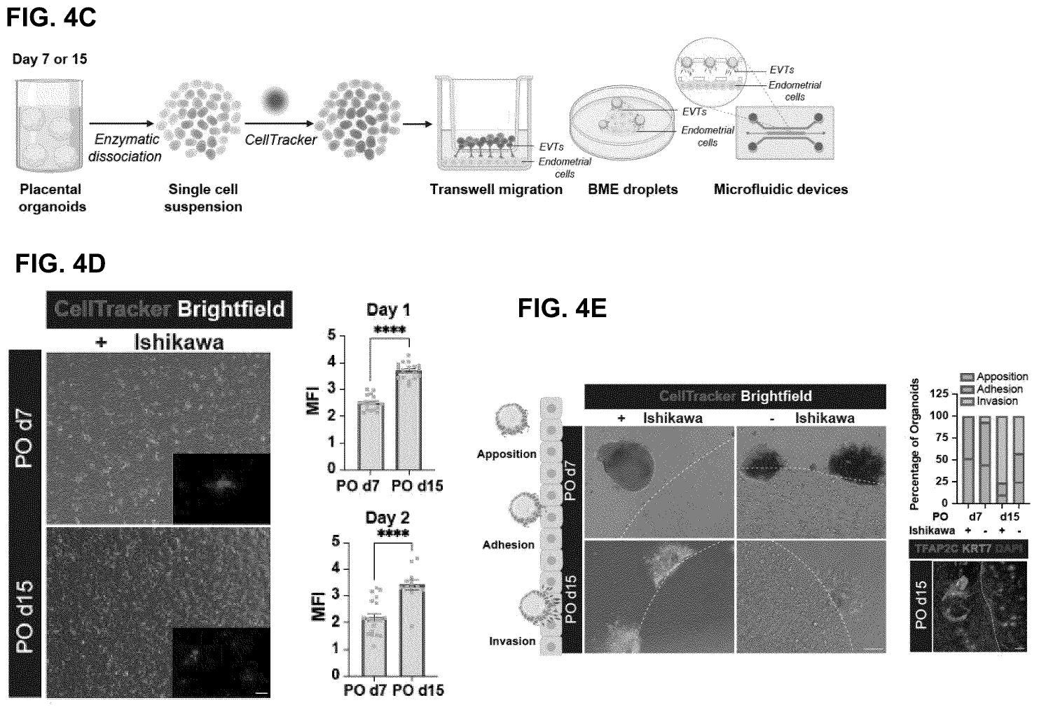

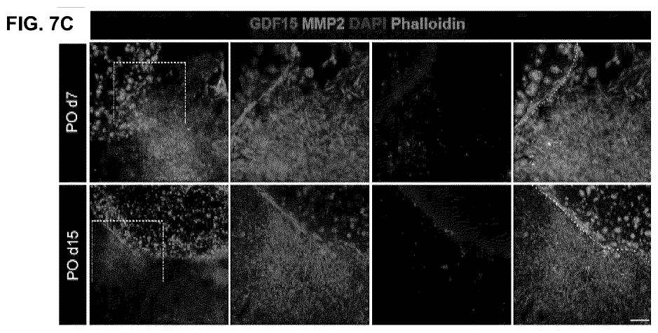

Implantation is a complex process involving sequential apposition, adhesion and invasion into the maternal endometrium. To investigate the ability of trophoblast containing placental organoids to attach and invade the endometrium (Ishikawa cells), experiments were performed in Transwell and BME droplets. In the Transwell systems, the CellTracker labeled day 7 and day 15 placental organoid cells migrated towards the Ishikawa cells. However, in the absence of Ishikawa cells, EVT migration was not observed. The number of labeled migrating cells were found to be higher in day 15 placental organoids as indicated in FIG. 4D. Furthermore, given the Ishikawa cells present mixed characteristics of both glandular and luminal epithelium, it is representative of a receptive endometrium for the placenta implantation studies. BME encapsulated Ishikawa cells were found to facilitate the apposition, adhesion and invasion of CellTracker labeled placental organoids on the BME droplets (FIG. 4E). While most placental organoids adhered onto the BME droplets, a higher percentage of invading cells were observed in day 15 placental organoids. The migrating EVT cells in the BME with or without Ishikawa cells were further characterized by immunofluorescence (FIG. 7A, 7B, and 7C).

### Methods

Generation of Naïve Stem Cells from iPSCs

Human iPSCs were purchased from Gibco. iPSCs were cultured in feeder-free conditions on vitronectin XF™ (STEMCELL Technologies #100-0763)-coated plates in mTESR™ media (STEMCELL Technologies #85850) at 37° C. in 5% CO₂ and passaged using ReLeSR™ (STEMCELL Technologies #100-0484) every 5-8 days. The media was switched to RSeT™ media (STEMCELL Technologies #05978) to induce reprogramming of iPSCs into naïve stem cells. Cell colonies were monitored for compaction and defined edges. The naïve stem cell colonies were maintained in RSeT™ media for 5 days and characterized, while the iPSC controls were maintained in mTESR™1 media.

### Differentiation of Naïve Stem Cells to Placental Organoids

Naïve stem cells were washed with PBS and dissociated with ReLeSR™ to obtain a single cell suspension. The cell suspension was counted and seeded onto anti-adherence rinsing solution (STEMCELL Technologies #07010) treated AggreWell™ plates (STEMCELL Technologies #34415) at a seeding density of 500 cells/spheroid in either RSeT™ media (naïve stem cells) or mTESR™1 (iPSC controls) supplemented with 10 μM Y-27632 (Tocris #1254) and centrifuged at 300×g for 5 minutes to facilitate aggregate formation. After 2 days, the RSeT™ media or mTESR™1 was replaced by DMEM-F12 (STEMCELL Technologies #36254) containing 1×N2 (Gibco #17502048), 1×B-27 (Gibco #175024044), 50 ng/ml EGF (Peprotech #AF-100-15), 50 ng/mL HGF (Peprotech #100-39H), 80 ng/mL R-Spondin-1 (Peprotech #120-38), 100 ng/ml FGF-2(Peprotech #100-18B), 1.5 μM CHIR 99021 (Tocris #4423), 0.5 μM A83-01 (SelleckChem #S7692), 1 μM SB431542 (Tocris #1614), 2 μM Y-27632 (Tocris #1254) and 2.5 μM PGE2(SantaCruz Biotechnology #363-24-6) maintained for 7 days or 15 days in TSC differentiation media.

Cell culture

Human umbilical vein endothelial cells (HUVEC) expressing RFP (Angio-Proteomie) were cultured in endothelial growth medium 2 (EGM2, PromoCell) supplemented with 10% fetal bovine serum (FBS, Gibco). These cells were passaged or harvested for use at 70-80% confluence until passage 10.

Human dermal fibroblasts (HDFs) (PromoCell) were cultured in fibroblast growth medium 2 (FGM2, PromoCell) until 80% confluent, then passaged or utilized until passage 15.

Human endometrial adenocarcinoma, Ishikawa cell lines (Sigma-Aldrich) were cultured in minimal essential media (MEM, Gibco) supplemented with 2 mM Glutamine, 1% non-essential amino acids (NEAA) and 5% FBS until 70% confluence and passaged. The cells were maintained at 5% CO₂ in a 37° C. incubator.

### Immunofluorescence Staining

The adherent cells and organoids were washed twice with PBS and fixed with 4% paraformaldehyde (PFA) (SantaCruz Biotechnology #SC281692) for 15 minutes and 30 minutes respectively at room temperature. Upon fixation, the cells and organoids were washed thrice with PBS, permeabilized and blocked in PBS containing 0.5% BSA and 0.1% Triton X-100, for 1 hour at room temperature. The blocked cells or organoids were incubated in primary antibodies SOX2 (Cell Signaling Technologies #23064), TBX3 (SantaCruz Biotechnology #sc-166623), KRT7 (Cell Signaling Technologies #4465), TFAP2C (SantaCruz Biotechnology #sc-12762), MMP2 (Abcam #ab92536), HLA-G (Abcam #ab52454), FN1 (Cell Signaling Technologies #26836), GDF15 (Abcam #ab106112), and CD31 (Invitrogen #MA5-29474), overnight at 4° C., washed with PBS and incubated with secondary antibodies (ThermoFisher Scientific #A32731, #11003) for 1 hour room temperature in dark. Nuclei were counterstained with DAPI (ThermoFisher Scientific #62248) for 10 minutes in dark at room temperature. Immunofluorescent stained cells and organoids were then visualized using Keyence microscope.

### Cell Proliferation Assay

The proliferation of stem cells were detected using 5-ethynyl-2′-deoxyuridine (EdU) staining and flow cytometry using the EdU staining proliferation kit (abcam #ab222421) according to the manufacturer's instructions. Briefly, primed and naïve stem cells were seeded at an equal density and allowed to grow till ˜70% confluence. Cells were labelled with 15 mM EdU for 3 hours at 37° C. in 5% CO₂. After incubation, the cells were fixed with the fixative solution for 20 minutes at room temperature protected from light. After washing, the cells were permeabilized with permeabilization solution for 30 minutes at room temperature, followed by washes and the addition of EdU additive solution reaction buffer for 30 minutes at room temperature. The cells were then washed, counterstained with DAPI and imaged using the using Keyence microscope. Tryphan blue assay was also performed every day for a period of 5 days to estimate the proliferation rate of naïve vs. primed stem cells. Briefly, the cells were seeded at equal density, trypsinized, counted with a microscope by staining with Tryphan blue in a haemocytometer. This process was repeated daily for a period of 5 days and the cell count was plotted.

### RNA Isolation and Quantitative RT-PCR

Total RNA was isolated using the PureLink™ RNA mini kit (ThermoFisher Scientific #12183025) following the manufacturer's instructions. The RNA quality and concentration was assessed using Nanodrop. cDNA synthesis was performed on the total RNA using the High-Capacity cDNA Reverse Transcription Kit (ThermoFisher Scientific #4368814). Quantitative real-time PCR was performed using Taqman probes (Primers are listed in Table . . . ) on the QuantStudio 3 Real-Time PCR System (ThermoFisher Scientific #A28567). All the samples were run in triplicates among 3 biological replicates. The gene expression was normalized to housekeeping genes, GAPDH and TBP and fold changes were determined accordingly.

### hCG Secretion Analysis

The secretion of hCG by placental organoids was confirmed using an over-the-counter pregnancy test. The conditioned media from day 7 and day 15 placental organoids was collected, pooled and dropped on a pregnancy test strip. It was allowed to stand for 2 minutes and observed for the development of multiple bands to display a positive pregnancy test.

In addition, to quantify the hCG in the secretome of the day 7 and day 15 placental organoids, hCG ELISA was performed according to the manufacturer's instructions (Invitrogen #EH235RB). Briefly, standards and diluted samples were added to pre-coated wells and incubated at room temperature for 2.5 hours with gentle shaking. Post- incubation, the plate was washed thrice with 1× Wash buffer and blotted. Biotin conjugate was added to each well and incubated for 1 hour room temperature with gentle shaking. Then, the plate was washed thrice with 1× Wash buffer and blotted, followed by Streptavidin-HRP addition for 45 mins at room temperature in dark. The solution was discarded, the plate was washed thrice with 1× Wash buffer and blotted. Upon the addition of TMB substrate solution, over a period of 30 minutes in dark, the substrate turns blue. The reaction is then stopped with Stop solution and the absorbance of the plate is read at 450 nm using a Tecan plate reader.

### Flow Cytometry

To sort the live cells from dissociated placental organoids fluorescence activated cell sorting (FACS) was used. The day 7 and day 15 placental organoids were collected and washed with PBS. They were then dissociated using 0.2 U/mL DNase in Accutase (Gibco #A1110501) for 20 mins at 37° C. into single cell suspension. FACS buffer comprising of 2% BSA was then added and the single cells were centrifuged at 4° C. to obtain a pellet. Upon resuspension, the cells were stained with Zombie Aqua (1:200 dilution) (Biolegend #423101) and incubated for 10 mins in dark on ice. Post-staining the cells were washed and sorted for live cells using a flow cytometer (BD FACS Aria Fusion).

### Single Cell RNA Sequencing

iPSC spheroids, RSeT spheroids and placental organoids (day 7 and day 15) organoids were dissociated. The cells from dissociated organoids were sorted to enrich for live cells and washed with FACS buffer. Sample hashing with hashtag oligos enabled the pooling of multiple samples in a single tube. The pooled samples were resuspended in ice-cold PBS with 0.04% BSA at a final concentration of 2400 cells/mL. Then, the single cell suspensions were loaded onto a 10× Genomics Chromium Controller with a loading target of 30,000 cells. Using the Chromium Next Gen Single Cell 5′ Reagent Kit v2, libraries were generated according to the manufacturer's instructions, with additional steps to amplify the TotalSeq barcodes. The quality and quantity of libraries generated were measured on Bioanalyzer and sequenced on Illumina NovaSeq 6000 with a sequencing target of 30,000 reads per cell for gene expression libraries and 5000 reads per cell for the hashtag oligo libraries.

### Microfluidics Device Generation

The microfluidics devices used herein were fabricated as previously described, using standard soft lithography process, using SU-8 2100 (Kayaku) to form a 400 mm high layer on a silicon wafer. This wafer was then exposed to UV light through a photomask and developed to form the master mould. On casting polydimethylsiloxane (Slygard 184, Dow) onto the master mould and bonding irreversibly to glass-bottom 6-well plates using oxygen-plasma treatment for 60 seconds (Harrick). To promote gel adhesion, the channels of the devices are then coated with poly-L-lysine (0.01% w/v, 84 kDa, Millipore) and glutaraldehyde (1.0%, Sigma).

### Angiogenesis Assay

Using the 3-channel microfluidic device generated, an acellular collagen gel was seeded in the center channel. HUVECs were seeded in the top media channel at a density of 5×10⁶ cells/mL and the device was tilted for 10 minutes to allow cell attachment on the gel. After incubating the cells for 24 hours at 37° C. in 5% CO₂ HDFs were seeded at 2×10⁵ cells/mL in the lower media channel supplemented with 10% FBS and 50 ng/ml VEGF (Peprotech). Conditioned media (EGM control, TSC media control, Day 7 and 15 placental organoid conditioned) was added to the top channel with HUVECs. The devices were imaged daily and fixed with 4% PFA on day 2, immunostained, imaged and angiogenesis was quantified using ImageJ.

### Vasculogenesis Assay and Perfusion

HUVECs and HDFs were seeded at a density of 10×10⁶ cells/mL and 10×10⁴ cells/mL respectively. in the middle channel of the microfluidic device within a hydrogel composed of 5 mg/mL bovine fibrinogen (ThermoFisher), 10% basement membrane extract (Bio-Techne), and polymerized with bovine thrombin (ThermoFisher). After 24 hours the media was switched with conditioned media (EGM control, TSC media control, Day 7 and 15 placental organoid conditioned) and the vessel formation was observed for seven days. After three days of initial seeding, additional HUVECs were seeded on the side channels. To assess vessel functionality, 1 mm FITC-dextran GFP was permeated through the media channel.

### Angiogenesis Array

Placental organoids (day 7 and day 15) conditioned media was assessed for pro- and anti-angiogenic factors using the Proteome Profiler Human Angiogenesis Array Kit (R&D Systems #ARY007). Briefly, arrays and samples were prepared according to the manufacturer's recommendations. Using a streptavidin-HRP based system the signals were detected by chemiluminescence imaging and measured using ImageJ.

### Drug Toxicity Response

Placental organoids (12-15 organoids/well) were treated with different concentrations of nicotine (0.1, 0.5, 1, 5, 10, 20 mg/mL), acetaminophen (0.01, 0.1, 1, 5, 10, 20 mM), ibuprofen (25, 50, 100, 200, 400, 800 mg/mL) and lumiracoxib (0.01, 0.1, 1, 5, 10, 20 mM) for 24 and 48 hours to determine the IC₅₀ value for drug toxicity. Post-incubation with the drugs for 24 and 48 hours respectively, the cell viability was determined using a CellTiter-Glo Luminescent cell viability assay (Promega #G7570). Briefly, cell supernatant was mixed with equal volume of CellTiter-Glo reagent for 2 minutes on an orbital shaker, incubated for 10 minutes in dark at room temperature and then the luminescence was determined using a Tecan plate reader (Tecan Spark).

### Implantation Assay

For EVT invasion studies using Transwell plates, the day 7 and 15 placental organoids were enzymatically dissociated into a single cell suspension using Accutase treatment and labelled with Calcien-AM according to the manufacturer's instructions. The labelled cells were seeded onto the upper chamber of the Transwell (insert with pore size 8 mm) at a seeding density of 5×10⁴ cells/well and Ishikawa cells were seeded into the lower chamber at a cell density of 4×10⁴ cells/well. The lower chamber was imaged daily for 3 days to monitor and quantify the migrating EVT cells in the presence/absence of Ishikawa cells. To recapitulate and demonstrate implantation on endometrial wall, the Ishikawa cells were mixed with BME and dome shaped BME droplets were generated on cell culture plates. After 30 minute incubation, media was added so as to not disturb the BME droplets and placental organoids were added to the media. Placental organoids were added to the wells and the attachment and migration of EVT cells into the endometrial BME droplets in the presence/absence of Ishikawa cells were imaged and quantified.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed:

1. A placental organoid composition comprising a plurality of trophoblast cells derived from naïve-like stem cells in suspension cultures,
wherein the organoid exhibits a three-dimensional architecture resembling human placental tissue; and wherein the organoid demonstrates functional properties characteristic of human placental tissue, including expression of human leukocyte antigen-G (HLA-G), and the secretion of pregnancy related hormones.

2. The composition of claim 1, wherein the organoid further expresses transcription factor AP-2 Gamma (TFAP2C) and Keratin 7 (KRT7).

3. The composition of claim 1, wherein the pregnancy related hormones comprise GDF-15, hCG, or a combination thereof.

4. The composition of claim 1, wherein the trophoblast cells differentiate into cytotrophoblasts, syncytiotrophoblasts, and extravillous trophoblasts.

5. The composition of claim 4, wherein the syncytiotrophoblasts express Syndecan-1 (SDC1), Choriogonadotropin subunit beta (CGB), or a combination thereof.

6. The composition of claim 5, wherein the extravillous trophoblasts express HLA-G, growth differentiation factor 15 (GDF15), Matrix Metallopeptidase 2 (MMP2), Fibronectin 1 (FN1), or a combination thereof.

7. A method of assessing drug toxicity, the method comprising:
a) administering a drug to the placental organoid composition according to claim 1 for a period of time; and
b) measuring cell viability within the placental organoid composition.

8. The method of claim 7, wherein the drug comprises nicotine, acetaminophen, ibuprofen, lumiracoxib, or a combination thereof.

9. The method of claim 7, wherein the period of time is 24 hours or 48 hours.

10. The method of claim 7, wherein the drug is toxic if the cell viability is reduced below a predetermined threshold.

11. The method of claim 7, wherein the cell viability within the placental organoid composition is measured using a viability assay; wherein the drug is toxic if the cell viability is reduced below a predetermined threshold.

12. The method of claim 11, wherein the viability assay is a luminescence-based viability assay.

13. The method of claim 11, wherein the drug is toxic if the cell viability is reduced to less than 50%.

14. A method of generating a placental organoid composition, the method comprising:
a) obtaining a single-cell suspension of naïve-like stem cells;
b) seeding the single-cell suspension onto a culture surface in a first medium;
c) centrifuging the single-cell suspension to facilitate aggregate formation;
d) incubating the single cell suspension to form aggregates; and
e) replacing at least a portion of the first media with a differentiation media and incubating the aggregates for a period of time;
wherein the aggregates differentiate into placental organoids during incubation.

15. The method of claim 14, wherein the single-cell suspension is seeded at a density of about 500 cells/spheroid.

16. The method of claim 14, wherein the single-cell suspension is centrifuged at about 100×g to 300×g.

17. The method of claim 14, wherein the aggregates are incubated in the differentiation media for about 7 days.

18. The method of claim 14, wherein the aggregates are incubated in the differentiation media for about 15 days.

19. A kit for generating placental organoids, the kit comprising:
a) a medium for culturing a single-cell suspension of naïve-like stem cells; and
b) a differentiation medium for inducing the differentiation of the stem cells into trophoblasts.

20. The kit of claim 19, further comprising instructions for generating the placental organoid, wherein the instructions describe the steps for culturing the naïve-like stem cells in suspension to generate placental organoids comprising cytotrophoblasts, syncytiotrophoblasts, and extravillous trophoblasts.
